(19) United States
(12) Patent Application Publication
(10) Pub. No.: US 2025/0006322 A1
(43) Pub. Date: Jan. 2, 2025

(54) NUTRITION AND DEPRESCRIPTION

(71) Applicant: CVS Pharmacy, Inc., Woonsocket, RI (US)

(72) Inventors: Richard J. Wood, Barrington, RI (US); Sara Gregory-Dunne, Glastonbury, CT (US); Cameron James Smith, Mesa, AZ (US)

(21) Appl. No.: 18/757,266

(22) Filed: Jun. 27, 2024

Related U.S. Application Data
(60) Provisional application No. 63/523,816, filed on Jun. 28, 2023.

(51) Int. Cl. G16H 10/60; G16H 20/60

(52) U.S. Cl. CPC G16H 10/60; G16H 20/60

(57) ABSTRACT

A method, system, and apparatus are provided that include: receiving an electronic record including health information that is associated with an individual and receiving historical data about the health information including changes in the health information recorded over time. The method, system, and apparatus further include determining, based on the historical data and the changes in the health information recorded over time received, that a change to at least one of a structured diet plan, a lifestyle behavior, and a prescribed drug is available for the individual, and updating the electronic record to store the change.

### CROSS REFERENCE TO RELATED APPLICATION

The present application claims the benefit of and priority under 35 U.S.C. § 119 (e) to 63/523,816, filed on Jun. 28, 2023, the entire disclosure of which is hereby incorporated by reference.

### FIELD OF THE DISCLOSURE

The disclosure relates to systems and methods for monitoring and treatment of chronic diseases (e.g., Type 2 diabetes) of a patient using nutritional therapy, healthcare provider and artificial intelligence (AI) based coaching, patient self-management, lifestyle management, and medication alteration (e.g., deprescription).

### BACKGROUND

In healthcare, people with chronic conditions are hospitalized more frequently than those without. Techniques for treating patients having a chronic disease (e.g., Type 2 diabetes) are desired.

### SUMMARY

A method, including: receiving an electronic record including health information that is associated with an individual, the health information including fields of the electronic record including prescription-based electronic data, claims-based electronic data, a health condition associated with the individual, a prescribed drug associated with the individual that treats the health condition, a lifestyle behavior associated with the individual, and a structured diet plan associated with the individual; receiving historical data about the health information including changes in the health information recorded over time, wherein the changes in the health information include a change in a measured biomarker; determining, based on the historical data and the changes in the health information recorded over time received, that a change to at least one of the structured diet plan, the lifestyle behavior, and the prescribed drug is available for the individual; and updating the electronic record to store the change to the at least one of the structured diet plan, the lifestyle behavior, and the prescribed drug.

In some aspects, the method further includes: sending, across a communication network, an update message to a pharmacy or a provider based on determining that the change to at least one of the structured diet plan, the lifestyle behavior, and the prescribed drug is available for the individual.

In some aspects, the update message includes an instruction to alter a parameter associated with at least one of: the structured diet plan, the lifestyle behavior, and the prescribed drug.

In some aspects, the instruction alters at least one of a number of times a prescribed food should be consumed in a given time period, an amount of the prescribed food that should be consumed in the given time period, and a type of the prescribed food that should be consumed in the given time period.

In some aspects, the instruction includes a drug titration to alter the dosage for the prescribed drug associated with the individual, wherein the drug titration decreases at least one of a number of times the prescribed drug should be taken in a given time period and an amount of the prescribed drug that should be taken in the given time period.

In some aspects, the instruction alters at least one of a number of times a prescribed lifestyle action should be performed in a given time period, a duration of the prescribed lifestyle action that should be performed in the given time period, and a type of the prescribed lifestyle action that should be performed in the given time period.

In some aspects, determining the change to the prescribed drug is available includes determining that a drug titration of the prescribed drug is available for the individual.

In some aspects, determining that the drug titration of the prescribed drug is available for the individual includes: determining, based on a length of time since the individual was initially prescribed the prescribed drug exceeding a predetermined length, that at least one of a drug dependency test and a drug dependency evaluation is recommended for the individual; and at least one of: sending a text message to a communication device of the individual instructing the individual to obtain the drug dependency test; and sending an evaluation message to a communication device of a pharmacy or provider instructing the pharmacy or provider to generate the drug dependency evaluation, wherein the drug dependency evaluation includes an indication of a pattern, a trend, or both associated with the individual and the prescribed drug.

In some aspects, determining that the drug titration of the prescribed drug is available for the individual further includes: receiving results of a drug dependency test associated with the individual; determining, when the results of the drug dependency test indicate the individual is dependent on a minimum dosage of the prescribed drug, that the drug titration will not reach a zero amount of the prescribed drug within a predetermined period of time; and determining, when the results of the drug dependency test indicate the individual is not dependent on the minimum dosage of the prescribed drug, that the drug titration can reach a zero amount of the prescribed drug within the predetermined period of time.

In some aspects, determining that the change to at least one of the structured diet plan, the lifestyle behavior, and the prescribed drug is available for the individual includes: determining, based on the historical data, that a blood measurement of the individual is maintained within predetermined acceptable health levels over a predetermined period of time.

In some aspects, the method further includes: receiving, after the individual has adhered to the change to at least one of the structured diet plan, the lifestyle behavior, and the prescribed drug for a predetermined amount of time, a subsequent electronic record including subsequent health information associated with the individual, the subsequent health information including subsequent fields of the electronic record including at least one of blood data and weight information for the individual over the predetermined amount of time; and determining, based on the subsequent health information, that a further change to at least one of the structured diet plan, the lifestyle behavior, and the prescribed drug is available for the individual.

In some aspects, the further change to the prescribed drug includes a drug titration of the prescribed drug, wherein the drug titration includes reducing an altered dosage amount to zero when the individual is not dependent on a minimum dosage of the prescribed drug.

In some aspects, prior to receiving the electronic record, the method further includes: determining, based on the health information, the structured diet plan associated with the individual; and sending, across a communication network, the structured diet plan to a communication device of the individual causing the structured diet plan to be rendered by a display of the communication device of the individual.

In some aspects, the structured diet plan is generated by: providing a dataset to a machine learning model, wherein the dataset includes at least one of the prescription-based electronic data and the claims-based electronic data; and receiving an output from the machine learning model in response to the machine learning model processing at least a portion of the dataset, wherein the output includes the structured diet plan.

In some aspects, the dataset further includes at least one of: second prescription-based electronic data associated with the individual; second claims-based electronic data associated with the individual; biomarker data; and answers provided by the individual in response to score-weighted questions.

In some aspects: the structured diet plan is determined based on answers provided by the individual in response to score-weighted questions; the answers are provided in electronic form from a communication device of the individual; and the structured diet plan includes a food regimen, the food regimen including a diet based on one of low-carbohydrate foods, Mediterranean foods, dietary approaches to stop hypertension (DASH) foods, and plant-based foods that eliminate meat consumption.

In some aspects, determining that the change to at least one of the structured diet plan, the lifestyle behavior, and the prescribed drug is available for the individual is based on answers provided by the individual in response to score-weighted questions.

A system for reducing medication dependency, including: a communications interface; a processor coupled with the communications interface; and a memory coupled with the processor, wherein the memory stores data that, when executed by the processor, enables the processor to: receive, via the communications interface, an electronic record including health information that is associated with an individual, the health information including fields of the electronic record including prescription-based electronic data, claims-based electronic data, a health condition associated with the individual, a prescribed drug associated with the individual that treats the health condition, a lifestyle behavior associated with the individual, and a structured diet plan associated with the individual; receive, via the communications interface, historical data about the health information including changes in the health information recorded over time, wherein the changes in the health information include a change in a measured biomarker; determine, based on the historical data and the changes in the health information recorded over time received, that a change to at least one of the structured diet plan, the lifestyle behavior, and the prescribed drug is available for the individual; and update the electronic record to store the change to the at least one of the structured diet plan, the lifestyle behavior, and the prescribed drug.

In some aspects, the data, when executed by the processor, further enables the processor to: send, via the communications interface, an update message to a pharmacy or a provider based on determining that the change to at least one of the structured diet plan, the lifestyle behavior, and the prescribed drug is available for the individual.

In some aspects, the update message includes an instruction to alter a parameter associated with at least one of: the structured diet plan, the lifestyle behavior, and the prescribed drug.

In some aspects, determining the change to the prescribed drug is available includes determining that a drug titration of the prescribed drug is available for the individual, wherein in determining that the drug titration of the prescribed drug is available for the individual, the data, when executed by the processor, further enables the processor to: determine, based on a length of time since the individual was initially prescribed the prescribed drug exceeding a predetermined length, that at least one of a drug dependency test and a drug dependency evaluation is recommended for the individual; and at least one of: send, via the communication interface, a text message to a communication device of the individual instructing the individual to obtain the drug dependency test; and send, via the communication interface, an evaluation message to a communication device of a pharmacy or provider instructing the pharmacy or provider to generate the drug dependency evaluation, wherein the drug dependency evaluation includes an indication of a pattern, a trend, or both associated with the individual and the prescribed drug.

A computer-implemented method, including: receiving, at a device, an electronic record including health information that is associated with an individual, the health information including fields of the electronic record including: prescription-based electronic data; claims-based electronic data; a health condition associated with the individual; a prescribed drug associated with the individual that treats the health condition; lifestyle behavior associated with the individual; and a structured diet plan associated with the individual; receiving, at the device, historical data about the health information including changes in the health information recorded over time, wherein the changes in the health information include a change in a measured biomarker; determining, at the device, based on the historical data and the changes in the health information recorded over time received, that a change to at least one of the structured diet plan, the lifestyle behavior, and the prescribed drug is available for the individual, wherein the change to the prescribed drug includes a drug titration; and updating the electronic record to store the change to the at least one of the structured diet plan, the lifestyle behavior, and the prescribed drug.

In some aspects, the method further includes: training a machine learning model in a first training stage based on a first training set, the first training set including a first set of feature vectors of a first set of individuals for which adherence to one or more prescribed actions achieved one or more relatively positive impacts in association with the health condition; and training the machine learning model in a second training stage based on a second training set, the second training set including a second set of feature vectors of a second set of individuals for which adherence to the one or more prescribed actions failed to achieve the one or more relatively positive impacts in association with the health condition, wherein determining that at least one of the structured diet plan, the lifestyle behavior, and the prescribed drug is available for the individual includes processing at least a portion of the electronic record and at least a portion of the historical data using the machine learning model.

In some aspects, determining that at least one of the structured diet plan, the lifestyle behavior, and the prescribed drug is available for the individual includes: providing the electronic record and the historical data to a machine learning model; and receiving an output from the machine learning model in response to the machine learning model processing at least a portion of the electronic record and at least a portion of the historical data, wherein the output includes an indication of the change to at least one of the structured diet plan, the lifestyle behavior, and the prescribed drug.

All examples and features mentioned above can be combined in any technically possible way.

### BRIEF DESCRIPTION OF THE DRAWINGS

The present disclosure is described in conjunction with the appended figures, which are not necessarily drawn to scale:

FIG. 1 illustrates an example of a system that supports aspects of the present disclosure.

FIG. 2 illustrates examples of user interfaces in accordance with aspects of the present disclosure.

FIG. 3 illustrates examples of user interfaces in accordance with aspects of the present disclosure.

FIG. 4 illustrates examples of user interfaces in accordance with aspects of the present disclosure.

FIG. 5 illustrates examples of user interfaces in accordance with aspects of the present disclosure.

FIG. 6 illustrates an example of an electronic record or report in accordance with aspects of the present disclosure.

FIGS. 7 and 8 illustrate example process flows that support aspects of the present disclosure.

### DETAILED DESCRIPTION

Before any examples of the disclosure are explained in detail, it is to be understood that the disclosure is not limited in its application to the details of construction and the arrangement of components set forth in the following description or illustrated in the following drawings. The disclosure is capable of other configurations and of being practiced or of being carried out in various ways. Also, it is to be understood that the phraseology and terminology used herein is for the purpose of description and should not be regarded as limiting.

While various examples of treating a medical condition (e.g., reversing the medical condition, reversing the progression of the medical condition) of a member will be described in connection with a member having diabetes (e.g., Type 1 diabetes, Type 2 diabetes, diabetes in pregnancy, etc.), it should be appreciated that the disclosure is not so limited. For instance, it is contemplated that examples of the present disclosure can be applied to treating (e.g., reversing) medical conditions of many different types. In other words, the framework described herein for reversing a medical condition can be leveraged to support management opportunities for any type or number of different medical conditions. Examples of such medical conditions that can be addressed or improved (e.g., reversed) with the framework described herein include, without limitation, prediabetes, cardiac conditions, heightened cholesterol, heightened blood pressure, hypertension, post-operative conditions, pre-operative conditions, cancer and other chronic conditions, infertility, chronic pain, broken bones, torn ligaments, torn muscles, etc. The terms “member”, “patient”, “user”, and “individual” may be used interchangeably herein.

Diabetes mellitus is a complex, progressive chronic condition in which the body's ability to produce or respond to the hormone insulin is impaired. Such impairment may result in abnormal metabolism of carbohydrates and elevated levels of glucose in the blood and urine of a patient.

Some approaches to achieving optimal diabetes control are hindered by high treatment cost associated with managing diabetes, and such approaches fail to provide for reducing a member's reliance on corresponding medication for managing diabetes. That is, for example, some approaches for managing diabetes fail to support programs (e.g., complementary to primary care physician (PCP) care) into which a member may enroll for diabetes reversal. Accordingly, for example, among members with a medical condition (e.g., Type 2 diabetes), some healthcare management systems that provide for managing the medical condition are ineffective at reversing the medical condition.

Machine learning and deep learning-based approaches have built upon the abilities of clinicians to assess current complications and predict the future health conditions of a member. However, while some machine learning approaches may yield relatively high accuracy with respect to assessing and predicting the future health conditions of a member, clinical applications of these approaches have been scarce due to an inability of some systems to translate model findings to actionable treatment (e.g., for reversing a medical condition).

U.S. Pat. No. 10,319,477 (Bill, D., “Dynamic data-driven biological state analysis”) describes generating recommendations based on user information. Bill describes automatically adjusting a dosage for a prescribed drug. Bill further describes making changes to a treatment plan based on biomarker data. Bill further describes retrieving medical data stored in a patient record in association with generating treatment plans and nutritional recommendations.

U.S. Patent Publication 2021/0174924 (Iyer, et al., “Digital therapeutic systems and methods”) describes features for identifying target users for a digital therapeutic (a mobile health (mHealth) application) based on one or more target parameters, conducting outreach to one or more of the target users using an outreach medium, identifying an activation mechanism to optimize use of the digital therapeutic, and activating the digital therapeutic for the target users based on the activation mechanism. Iyer describes applying the digital therapeutic for preventing, managing, or treating disease. Iyer includes further descriptions for determining targets for the digital therapeutic based on clinical factors, disease factors, technology or technography factors, social factors, and/or demographic factors. Iyer further describes determining outreach to targets.

U.S. Patent Publication 2021/0249133 (Mcraith, et al., “Database management and graphical user interfaces for measurements collected by analyzing blood”) describes features for database management and graphical user interfaces for measurements collected by analyzing blood. Mcraith describes generating treatment plans combining dosage and diet data. Mcraith describes techniques for reducing treatment window and for reducing or eliminating intake of specific food groups. In Mcraith, a provider may increase or decrease dosages of medication. The provider may update a titration regimen. Mcraith further describes retrieving and updating user electronic medical records in association with generating treatment plans. Mcraith provides examples of increasing dosage and fasting meals to lower blood glucose.

Approaches capable of offering tangible implementations to actionable member guidance and treatment (e.g., nutritional therapy, lifestyle adjustments, medication deprescription, etc.) are desired. For example, other approaches do not describe techniques inclusive of tapering of dosages to wean a patient off of medication (e.g., medication deprescription, decreasing dosage, etc.). Further, other approaches fail to consider claims data in evaluating a treatment plan. In addition, other approaches do not evaluate macronutrients and granular diet data.

The techniques described herein support AI based coaching and medication alteration (e.g., deprescribing, deescalation, titration, decreasing dosage amounts, decreasing dosage frequency, etc.) which may improve medical conditions of a member. For example, machine learning techniques described herein support AI based coaching, nutrition alteration, lifestyle management, and/or medication alteration which may reverse medical conditions of a member and/or prevent medical conditions from worsening. In some aspects, the machine learning techniques described herein consider claims data, macronutrients, and/or granular diet data in evaluating a treatment plan, which may thereby provide increased accuracy in determining a holistic treatment plan (e.g., including nutrition alteration, lifestyle management, medication alteration, and the like) for reversing the medical conditions of the member and/or prevent the medical conditions from worsening.

Example aspects of the present disclosure are described with reference to the following figures.

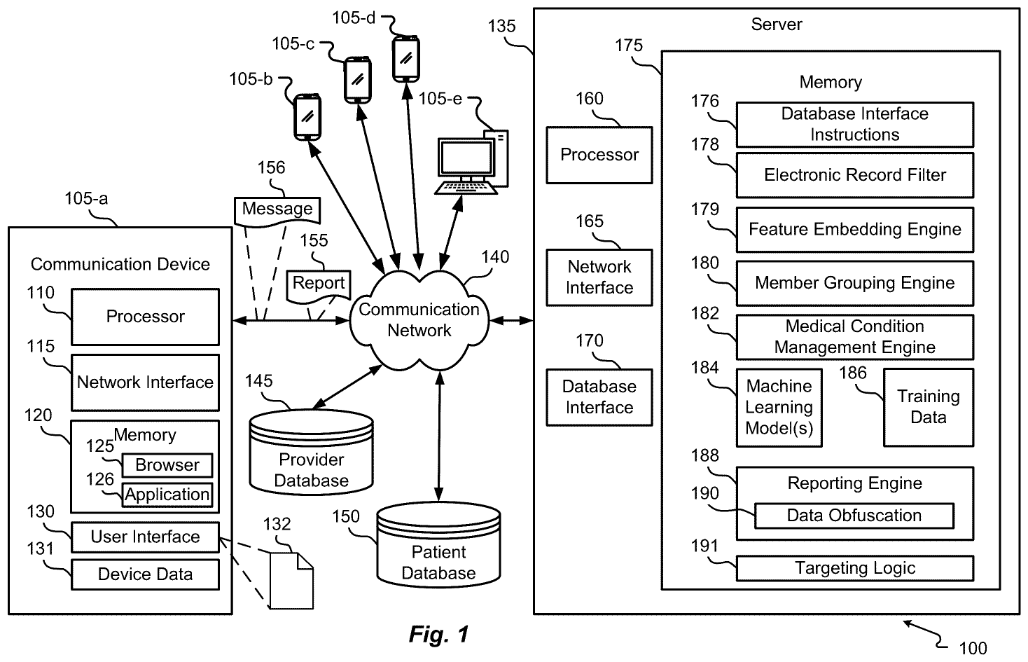

FIG. 1 illustrates an example of a system 100 that supports AI based coaching, nutrition therapy, lifestyle management, and medication alteration in association with treating medical conditions (e.g., diabetes, blood pressure, chronic pain, etc.) in accordance with aspects of the present disclosure. The system 100, in some examples, may include one or more computing devices operating in cooperation with one another to analyze electronic records (e.g., health information) of a member, analyze historical data about the health information, and analyze member behavior. The system 100 may determine a holistic approach including nutritional therapy, lifestyle managements, and/or medication alterations (e.g., deprescribing, deescalation, titration, etc.) for treating a medical condition of the member, and provide AI based coaching in association with treating the medical condition. In some other aspects, the system 100 may support generation of reports based on which a holistic plan (e.g., nutritional therapy, lifestyle management, and/or medication alteration) may be determined by a healthcare provider. In some aspects, the system 100 may support autonomous and/or semi-autonomous determination of a holistic plan by a device of the system 100. The system 100 may be, for example, a healthcare management system.

The components of the system 100 may be utilized to facilitate one, some, or all of the methods described herein or portions thereof without departing from the scope of the present disclosure. Furthermore, the servers described herein may include example components or instruction sets, and aspects of the present disclosure are not limited thereto. In an example, a server may be provided with all of the instruction sets and data depicted and described in the server of FIG. 1. Alternatively, or additionally, different servers or multiple servers may be provided with different instruction sets than those depicted in FIG. 1.

The system 100 may include communication devices 105 (e.g., communication device 105-a through communication device 105-c), a server 135, a communication network 140, a provider database 145, and a member database 150. The communication network 140 may facilitate machine-to-machine communications between any of the communication device 105 (or multiple communication devices 105), the server 135, or one or more databases (e.g., a provider database 145, a member database 150). The communication network 140 may include any type of known communication medium or collection of communication media and may use any type of protocols to transport messages between endpoints. The communication network 140 may include wired communications technologies, wireless communications technologies, or any combination thereof.

The Internet is an example of the communication network 140 that constitutes an Internet Protocol (IP) network consisting of multiple computers, computing networks, and other communication devices located in multiple locations, and components in the communication network 140 (e.g., computers, computing networks, communication devices) may be connected through one or more telephone systems and other means. Other examples of the communication network 140 may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VOIP) network, a cellular network, and any other type of packet-switched or circuit-switched network known in the art. In some cases, the communication network 140 may include of any combination of networks or network types. In some aspects, the communication network 140 may include any combination of communication mediums such as coaxial cable, copper cable/wire, fiber-optic cable, or antennas for communicating data (e.g., transmitting/receiving data).

A communication device 105 (e.g., communication device 105-a) may include a processor 110, a network interface 115, a computer memory 120, a user interface 130, and device data 131. In some examples, components of the communication device 105 (e.g., processor 110, network interface 115, computer memory 120, user interface 130) may communicate over a system bus (e.g., control busses, address busses, data busses) included in the communication device 105. In some cases, the communication device 105 may be referred to as a computing resource. The communication device 105 may establish one or more connections with the communication network 140 via the network interface 115. In some cases, the communication device 105 may transmit or receive packets to one or more other devices (e.g., another communication device 105, the server 135, the provider database 145, the provider database 150) via the communication network 140.

Non-limiting examples of the communication device 105 may include, for example, personal computing devices or mobile computing devices (e.g., laptop computers, mobile phones, smart phones, smart devices, wearable devices, tablets, etc.). In some examples, the communication device 105 may be operable by or carried by a human user. In some aspects, the communication device 105 may perform one or more operations autonomously or in combination with an input by the user. In some aspects, the communication device 105 may be coupled (e.g., electronically, via a wired or wireless connection) to or integrated with a biometric measurement device (not illustrated) such as a blood glucose meter, a blood pressure monitor, etc.

The communication device 105 may support one or more operations or procedures associated with AI based coaching, nutrition therapy, lifestyle management, and medication alteration applicable to treating (e.g., reversing) a medical condition of a member. For example, the communication device 105 may support communications between multiple entities such as a healthcare provider, a medical insurance provider, a pharmaceutical distributor (e.g., a pharmacist), a member (e.g., a patient), or combinations thereof. In some cases, the system 100 may include any number of communication devices 105, and each of the communication devices 105 may be associated with a respective entity.

The communication device 105 may render or output any combination of notifications, messages, reports, menus, etc. based on data communications transmitted or received by the communication device 105 over the communication network 140. The communication device 105 may output any combination of visual, audible, or haptic alerts in association with transmitted communications, received communications, and/or user interaction with the communication device 105.

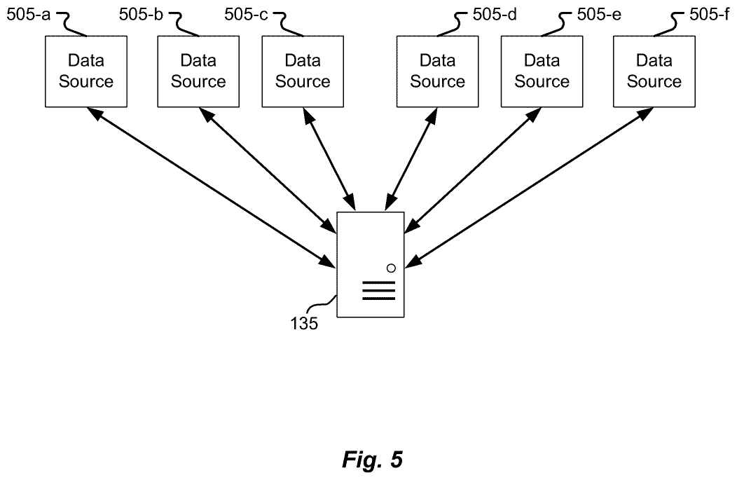

For example, the communication device 105 may receive one or more reports 155 (e.g., from the server 135) via the communication network 140. In an example, a report 155 may include an electronic record including health information associated with a member. Example aspects of the electronic record are described with reference to FIG. 5. In some aspects, the report 155 may include historical data about the health information, example aspects of which are described herein. Additionally, or alternatively, the communication device 105 may receive (e.g., access) the electronic record and/or historical data from the server 135, separate from report 155.

In an example, the server 135 may generate and/or provide (e.g., electronically, via fax, via physical mail, etc.) the report 155 based on a schedule (e.g., periodically, semi-periodically, based on a configured schedule, etc.). For example, the server 135 may generate and/or provide the report 155 at a daily interval, a monthly interval, or the like. Additionally, or alternatively, the server 135 may generate and/or provide the report 155 according to a relatively higher frequency interval or a relatively lower frequency interval.

The report 155 may include results from laboratory tests ordered by an entity associated with the healthcare management system. In some examples, the report 155 may include a comparison of the results to a set (or sets) of previous results (e.g., results provided in previous reports 155). In an example, the report 155 may indicate alterations to treatment plans and/or medications prescribed to a member. In some examples, the report 155 may include adherence to the treatment plans and/or the medications. The treatment plans described herein may include any combination of nutritional treatment plans (e.g., a structured diet plan, a food regimen, etc.), lifestyle treatment plans (e.g., increasing the amount of rest each day, refraining from smoking, etc.), and/or medication treatment plans (e.g., prescribed dosages, prescribed medication type, etc.), but are not limited thereto.

In another example, the communication device 105 may generate and transmit one or more messages 156 (e.g., treatment update messages described herein) to another communication device 105. Additionally, or alternatively, the communication device 105 may generate a physical representation (e.g., a letter, a printout, etc.) of a message 156 which may be provided to the member, a healthcare provider, an insurance provider, a pharmacist, other personnel, or the like via a direct mail provider (e.g., postal service). A treatment update message may include an indication of updates to nutritional treatment plans, updates to lifestyle treatment plans, and/or updates to medication treatment plans (e.g., drug titration, etc.) as described herein.

In some aspects, the communication device 105 may render a presentation (e.g., visually, audibly, using haptic feedback, etc.) of the report 155 and/or the message 156 via the user interface 130. The user interface 130 may include, for example, a display, an audio output device (e.g., a speaker, a headphone connector), or any combination thereof. In some aspects, the communication device 105 may render a presentation using one or more applications (e.g., a browser application 125) stored on the memory 120. In an example, the browser application 125 may be configured to receive the report 155 (and/or a message 156) in an electronic format (e.g., in an electronic communication via the communication network 140) and present content of the report 155 (and/or a message 156) via the user interface 130.

The communication device 105 may render a presentation (e.g., visually, audibly, using haptic feedback, etc.) of an application (e.g., a browser application 125, application 126, etc.). In an example, the communication device 105 may render the presentation via the user interface 130. The user interface 130 may include, for example, a display (e.g., a touchscreen display), an audio output device (e.g., a speaker, a headphone connector), or any combination thereof. In some aspects, the applications (e.g., a browser application 125, application 126, etc.) may be stored on the memory 120. In some cases, the applications may include cloud-based applications or server-based applications (e.g., supported and/or hosted by the server 135). Settings of the user interface 130 may be partially or entirely customizable and may be managed by one or more users, by automatic processing, and/or by artificial intelligence.

In an example, any of the applications (e.g., browser application 125, application 126) may be configured to receive data in an electronic format and present content of data via the user interface 130. For example, the applications may receive data from another communication device 105, the server 135, or a database (e.g., provider database 145, patient database 150), and the communication device 105 may display the content via the user interface 130.

In some aspects, the server 135 may communicate the report 155 to a communication device 105 (e.g., communication device 105-a) of a member, a communication device 105 (e.g., communication device 105-b) of a healthcare provider, a communication device 105 (e.g., communication device 105-c) of an insurance provider, a communication device 105 (e.g., communication device 105-d) of a pharmacist or pharmacy, a communication device 105 (e.g., communication device 105-e) of other personnel, or the like. Additionally, or alternatively, the server 135 may communicate a physical representation (e.g., a letter, a printout, etc.) of the report 155 to the member, a healthcare provider, an insurance provider, a pharmacist, other personnel, or the like via a direct mail provider (e.g., postal service).

The provider database 145 and the member database 150 may include member electronic records (also referred to herein as a data records) stored therein. In some aspects, the electronic records may be accessible to a communication device 105 (e.g., operated by healthcare provider personnel, insurance provider personnel, a member, a pharmacist, etc.) and/or the server 135. In some aspects, a communication device 105 and/or the server 135 may receive and/or access the electronic records from the provider database 145 and the member database 150 (e.g., based on a set of permissions).

In some aspects, an electronic record associated with a member may include claims-based electronic data. For example, the electronic record may include electronic medical record (EMR) data. In another example, the claims-based electronic data may include data describing an insurance medical claim, pharmacy claim, and/or insurance claim made by the member and/or a medical provider. Accordingly, for example, the claims-based electronic data may come from providers or payers, and claims included in the claims-based electronic data may be of various types (e.g., medical, pharmacy, etc.).

In some other aspects, the electronic record associated with the member may include device data 131 obtained from a communication device 105 (e.g., communication device 105-a) associated with the member. For example, the device data 131 may include gyroscopic data, accelerometer data, beacon data, glucose readings, heart rate data, blood pressure data, blood oxygen data, temperature data, kinetics data, location data, motion data, a device identifier, and/or temporal data (e.g., a timestamp) measurable, trackable, and/or providable by the communication device 105 (or a device connected to the communication device 105) associated with the member.

In some aspects, the electronic record may include member preferences with respect to coaching (e.g., how the member likes to be coached). In some examples, the electronic record may include member answers associated with the questionnaire 132.

In some aspects, the electronic record may include data (e.g., laboratory data, biometric data/measurements, image data, etc.) based on which the server 135 (e.g., the medical condition management engine 182) may track targeted biomarkers. For example, the server 135 may track records of a member over time (e.g., in association with identifying changes in medical condition for a member). In some cases, the electronic record may include historical measurement data associated with biometrics such as blood glucose, blood pressure, heart rate, etc. In some cases, the electronic record may include diagnostic images such as magnetic resonance imaging (MRI) scans, computed tomography scans (CT), ultrasound images, X-ray images, or the like.

In an example, the electronic record may include information associated with a structured diet plan. For example, the electronic record may include member entries input via an application (e.g., a nutrition application, a food tracking application, an application associated with the digital health platform described herein, etc.) executed at the communication device 105. In some aspects, the information associated with the structured diet plan may be stored in the device data 131.

In accordance with aspects of the present disclosure, the device data 131 may be provided continuously, semi-continuously, periodically, and/or based on a trigger condition by the communication device 105 (e.g., a smart watch, a wearable monitor, a self-reporting monitor such as a glucometer, a smartphone carried by a user, etc.) around monitored parameters such as blood glucose, blood pressure, heart rate, etc. In some aspects, the device data 131 of a communication device 105 (e.g., communication device 105-a) may be referred to as “environmental data” associated with a user, which may be representative of aspects of environmental factors (e.g., lifestyle, socioeconomic factors, details about the environment, etc.) associated with a member.

Accordingly, for example, the electronic record may provide insurance claim information of a member, member specific behavior, generic insurance claim information (e.g., common to a set of members), and/or generic behaviors of member behavior (e.g., behavior common to a set of members). In some cases, the device data 131 may include wearable-device data, glucose readings, blood pressure readings, heart rate, body temperature, “invisible” data (e.g., device related information associated with a member, such as Bluetooth beacon information), and/or self-reporting monitored data (e.g., provided by self-reporting glucometers such as continuous glucose monitors (CGMs) that report kinetics).

In some aspects, the electronic record may include genetic data associated with a member. In some other aspects, the electronic record may include notes/documentation that is recorded at a communication device 105 in a universal and/or systematic format (e.g., subjective, objective, assessment, and plan (SOAP) notes/documentation) among medical providers, insurers, etc.

In some other aspects, the electronic records may be inclusive of aspects of a member's health history and health outlook. The electronic records may include a number of fields for storing different types of information to describe the member's health history and health outlook. As an example, the electronic records may include member identifier information such as, for example, name, address, member number, social security number, date of birth, etc. In some aspects, the electronic records may include treatment data such as, for example, member health history, member treatment history, lab test results (e.g., text-based, image-based, or both), pharmaceutical treatments and therapeutic treatments (e.g., indicated using predefined healthcare codes, treatment codes, or both), insurance claims history, healthcare provider information (e.g., doctors, therapists, etc. involved in providing healthcare services to the member), in-member information (e.g., whether treatment is associated with care), location information (e.g., associated with treatments or prescriptions provided to the member), family history (e.g., inclusive of medical data records associated with family members of the member, data links to the records, etc.), or any combination thereof. In some aspects, the electronic records may be stored or accessed according to one or more common field values (e.g., common parameters such as common healthcare provider, common location, common claims history, etc.). In some examples, an electronic record may include a defined health condition associated with the individual. The electronic record may include a prescribed drug associated with the individual that treats the defined health condition, a length of time since the individual was initially prescribed the prescribed drug, and a structured diet plan associated with the individual.

In some aspects, the provider database 145 may be accessible to a healthcare provider of a member (also referred to herein as a member), and in some cases, include member information associated with the healthcare provider that provided a treatment to the member. In some aspects, the provider database 145 may be accessible to an insurance provider associated with the member. The member database 150 may correspond to any type of known database, and the fields of the electronic records may be formatted according to the type of database used to implement the member database 150. Non-limiting examples of the types of database architectures that may be used for the member database 150 include a relational database, a centralized database, a distributed database, an operational database, a hierarchical database, a network database, an object-oriented database, a graph database, a NoSQL (non-relational) database, etc. In some cases, the member database 150 may include an entire healthcare history or journey of a member, whereas the provider database 145 may provide a snapshot of a member's healthcare history with respect to a healthcare provider. In some examples, the electronic records stored in the member database 150 may correspond to a collection or aggregation of electronic records from any combination of provider databases 145 and entities involved in the member's healthcare delivery (e.g., a pharmaceutical distributor, a pharmaceutical manufacturer, etc.).

The provider database 145 and/or the member database 150 may include chronic disease indicators recorded for each member using a database format associated with the provider database 145 and/or the member database 150. In some aspects, the provider database 145 and/or the member database 150 may support diagnosis and procedure codes classified according to the International Classification of Diseases 10th revision (ICD-10) and Current Procedure Terminology 4th revision (CPT-4) codes. In some aspects, the provider database 145 and/or the member database 150 may support the use of Generic Product Identifier (GPI) and National Drug Code (NDC) Directory information for common diabetes medications. The provider database 145 and/or member database 150 may include demographic information, including age, gender, race, and geography, identified using claims data. The provider database 145 and/or member database 150 may include data such as proportion of days covered (PDC), calculated as a ratio of the number of days in a period covered to the number of days in a given period for each member and corresponding medication.

The server 135 (e.g., using the medical condition management engine 182 described later herein) may support AI based coaching, nutrition therapy, lifestyle management, and medication alteration applicable to treating (e.g., reversing) a medical condition of a member with respect to the following example disease management domains: medication regimen optimization (e.g., (e.g., deprescribing, deescalation, titration, decreasing dosage amounts, decreasing dosage frequency, etc.), prescription adherence, preventative screenings and lifestyle management, and biometric monitoring (e.g., for diabetes, blood glucose monitoring and/or blood pressure monitoring). It should be noted that other medical conditions could be managed by tracking other related or applicable biomarkers.

The server 135 may include a processor 160, a network interface 165, a database interface 170, and a memory 175. In some examples, components of the server 135 (e.g., processor 160, a network interface 165, a database interface 170, and a memory 175) may communicate via a system bus (e.g., any combination of control busses, address busses, and data busses) included in the server 135. Aspects of the processor 160, network interface 165, database interface 170, and memory 175 may support example functions of the server 135 as described herein. For example, the server 135 may transmit packets to (or receive packets from) one or more other devices (e.g., one or more communication devices 105, another server 135, the provider database 145, the provider database 150) via the communication network 140. In some aspects, via the network interface 165, the server 135 may transmit database queries to one or more databases (e.g., provider database 145, member database 150) of the system 100, receive responses associated with the database queries, or access data associated with the database queries.

In some aspects, via the network interface 165, the server 135 may transmit one or more reports 155 and/or messages 156 (e.g., treatment update messages) described herein to one or more communication devices 105 of the system 100. The network interface 165 may include, for example, any combination of network interface cards (NICs), network ports, associated drivers, or the like. Communications between components (e.g., processor 160, network interface 165, database interface 170, and memory 175) of the server 135 and other devices (e.g., one or more communication devices 105, the provider database 145, the provider database 150, another server 135) connected to the communication network 140 may, for example, flow through the network interface 165.

The processors described herein (e.g., processor 110 of the communication device 105, processor 160 of the server 135) may correspond to one or many computer processing devices. For example, the processors may include a silicon chip, such as a Field Programmable Gate Array (FPGA), an Application-Specific Integrated Circuit (ASIC), any other type of Integrated Circuit (IC) chip, a collection of IC chips, or the like. In some aspects, the processors may include a microprocessor, a Central Processing Unit (CPU), a Graphics Processing Unit (GPU), or plurality of microprocessors configured to execute the instructions sets stored in a corresponding memory (e.g., memory 120 of the communication device 105, memory 175 of the server 135). For example, upon executing the instruction sets stored in memory 120, the processor 110 may enable or perform one or more functions of the communication device 105. In another example, upon executing the instruction sets stored in memory 175, the processor 160 may enable or perform one or more functions of the server 135.

The processors described herein (e.g., processor 110 of the communication device 105, processor 160 of the server 135) may utilize data stored in a corresponding memory (e.g., memory 120 of the communication device 105, memory 175 of the server 135) as a neural network. The neural network may include a machine learning architecture. In some aspects, the neural network may be or include one or more classifiers. In some other aspects, the neural network may be or include any machine learning network such as, for example, a deep learning network, a convolutional neural network, or the like. Some elements stored in memory 120 may be described as or referred to as instructions or instruction sets, and some functions of the communication device 105 may be implemented using machine learning techniques. In another example, some elements stored in memory 175 may be described as or referred to as instructions or instruction sets, and some functions of the server 135 may be implemented using machine learning techniques.

In some aspects, the processors (e.g., processor 110, processor 160) may support machine learning model(s) 184 which may be trained and/or updated based on data (e.g., training data 186) provided or accessed by any of the communication device 105, the server 135, the provider database 145, and the member database. The machine learning model(s) 184 may be built and updated by the medical condition management engine 182 based on the training data 186 (also referred to herein as training data and feedback). In some aspects, the training data 186 may include multiple training sets.

For example, the machine learning model(s) 184 may be trained with a first training set that includes feature vectors of members (e.g., accessed from provider database 145 or member database 150) for which adherence to one or more actions (e.g., a prescribed drug regimen, a deprescription plan, a structured diet plan, a food regimen, an exercise regimen, etc.) achieved one or more relatively positive impacts (e.g., positive cost impact, positive clinical impact, reversal of a medical condition, etc.).

In an example, the machine learning model(s) 184 may be trained with a second training set that includes feature vectors of members for which a failure to adhere to one or more actions (e.g., a prescribed drug regimen, a deprescription plan, a structured diet plan, a food regimen, an exercise regimen, etc.) resulted in a relatively negative impact (e.g., negative cost impact, negative clinical impact, progression to a negative medical diagnosis, etc.).

In another example, aspects of the present disclosure include training the machine learning model(s) 184 with a third training set that includes feature vectors of members (e.g., accessed from provider database 145 or member database 150) for which adherence to one or more actions (e.g., a prescribed drug regimen, a deprescription plan, a structured diet plan, a food regimen, an exercise regimen, etc.) still failed to achieve a target impact (e.g., a positive cost impact, a positive clinical impact, a reversal of a medical condition, etc. with respect to a set of target criteria). For example, the third training set may include additional factors (e.g., outside of adherence to the one or more actions) that correlate to the failure to achieve the target impact.

In some other examples, aspects of the present disclosure include creating a fourth training set based on data included in any of the first through third training sets. For example, generating the fourth training set may include identifying a relatively larger set of factors (e.g., member characteristics, environmental factors, actions, etc.) that may affect whether a target impact is achievable.

The machine learning model(s) 184 may be provided in any number of formats or forms. Example aspects of the machine learning model(s) 184, such as generating (e.g., building, training) and applying the machine learning model(s) 184, are described with reference to the figure descriptions herein.

Non-limiting examples of the machine learning model(s) 184 include Decision Trees, gradient-boosted decision tree approaches (GBMs), Support Vector Machines (SVMs), Nearest Neighbor, and/or Bayesian classifiers, and neural-network-based approaches.

In an example, the machine learning model(s) 184 may include ensemble classification models (also referred to herein as ensemble methods) such as gradient boosting machines (GBMs). Gradient boosting techniques may include, for example, the generation of decision trees one at a time within a model, where each new tree may support the correction of errors generated by a previously trained decision tree (e.g., forward learning). Gradient boosting techniques may support, for example, the construction of ranking models for information retrieval systems. A GBM may include decision tree-based ensemble algorithms that support building and optimizing models in a stage-wise manner.

According to example aspects of the present disclosure described herein, the machine learning model(s) 184 may include Gradient Boosting Decision Trees (GBDTs). Gradient boosting is a supervised learning technique that harnesses additive training and tree boosting to correct errors made by previous models, or regression trees.

The machine learning model(s) 184 may include extreme gradient boosting (CatBoost) models. CatBoost is an ensemble learning method based on GBDTs. In some cases, CatBoost methods may have improved performance compared to comparable random forest-based methods. CatBoost methods are easily tunable and scalable, offer a higher computational speed in comparison to other methods, and are designed to be highly integrable with other approaches including Shapley Additive Explanations (SHAP) values.

In some aspects, the machine learning model(s) 184 may include ensemble classification models (also referred to herein as ensemble methods) such as random forests. Random forest techniques may include independent training of each decision tree within a model, using a random sample of data. Random forest techniques may support, for example, medical diagnosis techniques described herein using weighting techniques with respect to different data sources.

Various example aspects of the machine learning model(s) 184, inputs to the machine learning model(s) 184, and the training data 186 with respect to the present disclosure are described herein.

The memory described herein (e.g., memory 120, memory 175) may include any type of computer memory device or collection of computer memory devices. For example, a memory (e.g., memory 120, memory 175) may include a Random Access Memory (RAM), a Read Only a Memory (ROM), a flash memory, an Electronically-Erasable Programmable ROM (EEPROM), Dynamic RAM (DRAM), or any combination thereof.

The memory described herein (e.g., memory 120, memory 175) may be configured to store instruction sets, neural networks, and other data structures (e.g., depicted herein) in addition to temporarily storing data for a respective processor (e.g., processor 110, processor 160) to execute various types of routines or functions. For example, the memory 175 may be configured to store program instructions (instruction sets) that are executable by the processor 160 and provide functionality of any of the engines (e.g., feature embedding engine 179, member grouping engine 180, medical condition management engine 182, reporting engine 188, etc.) described herein.

The memory described herein (e.g., memory 120, memory 175) may also be configured to store data or information that is useable or capable of being called by the instructions stored in memory. Examples of data that may be stored in memory 175 for use by components thereof include machine learning model(s) 184 and/or training data 186 described herein.

Any of the engines (e.g., feature embedding engine 179, member grouping engine 180, medical condition management engine 182, reporting engine 188, etc.) described herein may include a single or multiple engines.

With reference to the server 135, the memory 175 may be configured to store instruction sets, neural networks, and other data structures (e.g., depicted herein) in addition to temporarily storing data for the processor 160 to execute various types of routines or functions. The illustrative data or instruction sets that may be stored in memory 175 may include, for example, database interface instructions 176, an electronic record filter 178 (also referred to herein as a feature vector filter), a feature embedding engine 179, a medical condition management engine 182, and a reporting engine 188. In some examples, the reporting engine 188 may include data obfuscation capabilities 190 via which the reporting engine 188 may obfuscate, remove, redact, or otherwise hide personally identifiable information (PII) from a report 155 (and/or electronic record) prior to transmission thereof to another device (e.g., communication device 105).

In some examples, the database interface instructions 176, when executed by the processor 160, may enable the server 135 to send data to and receive data from the provider database 145, the member database 150, or both. For example, the database interface instructions 176, when executed by the processor 160, may enable the server 135 to generate database queries, provide one or more interfaces for system administrators to define database queries, transmit database queries to one or more databases (e.g., provider database 145, the member database 150), receive responses to database queries, access data associated with the database queries, and format responses received from the databases for processing by other components of the server 135.

The server 135 may use the electronic record filter 178 in connection with processing data received from the various databases (e.g., provider database 145, member database 150). For example, the electronic record filter 178 may be leveraged by the database interface instructions 176 to filter or reduce the number of electronic records (e.g., feature vectors) provided to any of the feature embedding engine 179 or the medical condition management engine 182. In an example, the database interface instructions 176 may receive a response to a database query that includes a set of feature vectors (e.g., a plurality of feature vectors associated with different members). In some aspects, any of the database interface instructions 176, the feature embedding engine 179, or the medical condition management engine 182 may be configured to utilize the electronic record filter 178 to reduce (or filter) the number of feature vectors received in response to the database query, for example, prior to processing data included in the feature vectors.

The feature embedding engine 179 may receive, as input, sequences of medical terms extracted from claim data (e.g., medical claims, pharmacy claims) for each member. In an example, the feature embedding engine 179 may process the input using neural word embedding algorithms such as Word2vec. In some examples, the feature embedding engine 179 may process the input using Transformer algorithms (e.g., algorithms associated with language models such as Bidirectional Encoder Representations from Transformers (BERT) or Generative Pre-trained Transformer (GPT) or graph convolutional transformer (GCT)) and respective attentional mechanisms. In some aspects, based on the processing, the feature embedding engine 179 may compute and output respective dimension weights for the medical terms. In some aspects, the dimension weights may include indications of the magnitude and direction of the association between a medical code and a dimension. In an example, the feature embedding engine 179 may compute an algebraic average of all the medical terms for each member over any combination of dimensions (e.g., over all dimensions). In some aspects, the algebraic average may be provided by the feature embedding engine 179 as additional feature vectors in a predictive model described herein (e.g., classifier).

The member grouping engine 180, when executed by the processor 160, may enable the server 135 to group data records of various members according to a common value(s) in one or more fields of such data records. For example, the member grouping engine 180 may group electronic records based on commonalities in parameters such as medical conditions (e.g., diagnosis of diabetes, suggested actions associated with treating medical conditions, etc.), medical treatment histories, prescriptions, healthcare providers, locations (e.g., state, city, ZIP code, etc.), gender, age range, medical claims, pharmacy claims, lab results, medication adherence, demographic data, social determinants (also referred to herein as social indices), biomarkers, behavior data, engagement data, historical gap-in-care data, machine learning model-derived outputs, combinations thereof, and the like.

The reporting engine 188, when executed by the processor 160, may enable the server 135 to output one or more reports 155 (and/or provide electronic records described herein) based on data generated by any of the feature embedding engine 179, the member grouping engine 180, or the medical condition management engine 182. The reporting engine 188 may be configured to generate reports 155 (and/or provide electronic records) in various electronic formats, printed formats, or combinations thereof. Some example formats of the reports 155 (and/or electronic records) may include HyperText Markup Language (HTML), electronic messages (e.g., email), documents for attachment to an electronic message, text messages (e.g., SMS, instant messaging, etc.), combinations thereof, or any other known electronic file format. Some other examples include sending, for example, via direct mail, a physical representation (e.g., a letter) of the report 155 (and/or electronic records).

The reporting engine 188 may also be configured to hide, obfuscate, redact, or remove PII data from a report 155 prior to transmitting the report 155 to another device (e.g., a communication device 105). The reporting engine 188 may also be configured to hide, obfuscate, redact, or remove PII data from an electronic record prior to transmitting the electronic record to another device (e.g., a communication device 105). In some aspects, a communication device 105 may also be configured to hide, obfuscate, redact, or remove PII data from direct mail (e.g., a letter) prior to generating a physical representation (e.g., a printout) of a report 155 (and/or electronic record). In some examples, the data obfuscation may include aggregating electronic records to form aggregated member data that does not include any PII for a particular member or group of members. In some aspects, the aggregated member data generated by the data obfuscation may include summaries of data records for member groups, statistics for member groups, or the like.

According to example aspects of the present disclosure, a communication device 105 may support an interactive application (not illustrated) that begins with a questionnaire 132 including a set of questions (e.g., about 40-50 questions) designed to find a plan for treating a medical condition of a member (e.g., a best plan for reversing the medical condition). In an example, the application may be executed and/or displayed at the communication device 105. In some aspects, the application may be hosted at the server 135. Additionally, or alternatively, aspects of the present disclosure support providing a physical (e.g., paper) version of the questionnaire 132 to a member and digitally scanning (e.g., storing) the physical version to a memory (e.g., memory 120) of the communication device 105 or the server 135.

In some aspects, the questionnaire 132 may include multiple choice questions. In some other aspects, the questions may include survey questions (e.g., ranking questions) that ask a respondent to compare and rank items from a list in order of preference). In some other examples, the questions may be open ended, and the system 100 may support text analysis of provided answers (e.g., using a machine learning model(s) 184, using machine learning in association with natural language processing (NLP) and text analytics, using feature embedding engine 179, etc.).

Aspects of the present disclosure include providing data (e.g., answers) from the questionnaire 132 to the server 135 (e.g., to machine learning model(s) 184, to medical condition management engine 182, etc.) and/or to a communication device 105 of a healthcare provider. Accordingly, for example, the server 135 (e.g., machine learning model(s) 184, medical condition management engine 182) may provide an output associated with treating a medical condition (e.g., reversing a diabetes condition) of the member. Additionally, or alternatively, healthcare personnel (e.g., a physician, a nurse practitioner, a pharmacist, etc.) may provide clinical judgment and peer review in association with developing and/or providing the output.

The output (e.g., treatment plan) may include an indication of a nutrition pattern (e.g., a structured diet plan including a food regimen and/or an exercise regimen) determined as the best fit for the member. In some aspects, the nutrition pattern may be based on at least one of four diet types: a diet of low-carbohydrate foods, a diet of Mediterranean foods, a diet of dietary approaches to stop hypertension (DASH) foods, and a diet of plant-based foods (e.g., associated with eliminating meat consumption). In an example, the output may include an indication that the best diet for the member is Mediterranean, travel is an issue, and information (e.g., explanations) on why to cat certain things.

Determining the treatment plan may include an analysis (e.g., by the server 135, by healthcare personnel, etc.) of additional factors associated with the member. For example, determining the treatment plan may include determining likely behavioral factors (e.g., travel, weekends, peer pressure, etc.) associated with the member that may negatively or positively impact member adherence to the treatment plan. In another example, determining the treatment plan may be based on an analysis of scheduling data (e.g., calendar information, scheduling preferences, etc.) associated with the member. In some other examples, determining the treatment plan may be based on an analysis of psychosocial factors (e.g., characteristics) that may influence the member psychologically and/or socially into adhering to the treatment plan (or alternatively, failing to adhere to the treatment plan).

In some other aspects, determining the treatment plan may be based on an analysis (e.g., by the server 135, by healthcare personnel, etc.) of member preferences with respect to coaching (e.g., how the member likes to be coached). Some examples of member preferences with respect to coaching may include preferences corresponding to active text messaging, encouragements, motivational examples, etc., but are not limited thereto. In an example, the behavioral factors, scheduling data, psychosocial factors, and/or member preferences may be included in an electronic record described herein.

The output (e.g., treatment plan) may include an indication of a holistic approach to improve health. In some examples, the output may include an indication of nutrition alteration, lifestyle management, and/or medication alteration (e.g., deprescribing, deescalation, titration, decreasing dosage amounts, decreasing dosage frequency, etc.) to improve health. In some aspects, additional and/or alternative to an evaluation of caloric input and caloric output (e.g., calories in and calories out), aspects of the present disclosure support an analysis of nutrients (e.g., macronutrients, granular diet data, etc.) in determining nutritional therapy. Accordingly, for example, aspects of the present disclosure as described herein provide a combination of nutritional therapy (e.g., diet regimen), lifestyle management, and medication alteration (e.g., deprescription). In some examples, aspects of the present disclosure may include an analysis of claims/medical data (e.g., past claims) to determine biomarkers and preferences for members.

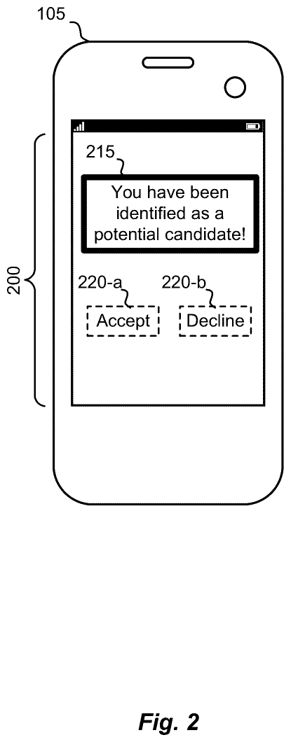

Other example illustrative aspects of the system 100 are described with reference to FIGS. 2 through 8.

FIG. 2 illustrates an example of a user interface 200 associated with the healthcare management system and digital health platform described herein. Example aspects of the user interface 200 and displaying notifications via the user interface 200 may be implemented by a communication device 105 and/or a server 135 described with reference to FIG. 1. For example, the user interface 200 may include aspects of user interface 130 of FIG. 1.

In an example described with reference to FIGS. 1 and 2, the server 135 may implement targeting logic 191 capable of determining (e.g., identifying, selecting, tracking, etc.) potential candidates for which the server 135 may provide a recommended diet and treatment plan. In some additional and/or alternative aspects, the targeting logic 191 may implement health and nutrition assessment and personalization techniques described herein in association with determining the potential candidates. Aspects of the targeting logic 191 may be implemented, for example, by executable instructions stored on a memory described herein (e.g., memory 120, memory 175). In some aspects, the targeting logic 191 may be implemented by a machine learning model(s) 184 described herein.

The server 135 (e.g., using the targeting logic 191) may determine the potential candidates from members or patients associated with the system 100. In some aspects, the server 135 may determine the potential candidates from among subsets of the members or patients, or the like. For example, the server 135 may divide the members or patients into subsets according to one or more criteria (e.g., age, demographics information, claims/medical data, existing or predicted medical conditions, social determinants of health (SDoHs), or the like).

The server 135 may determine the potential candidates based on information received from data sources unique to the system 100 (e.g., unique CVS data sources) in comparison to other systems. The data sources may include aspects of the provider database 145 and/or patient database 150. In some examples, the server 135 may retrieve, from the data sources, data such as user claims/medical data (e.g., including past claims, past diagnoses associated with a member, etc.). In some cases, the data sources may include questionnaire responses provided by the member or patient, in which the responses include a willingness of the member (or a degree of willingness) to be a candidate. In some other cases, the data sources may include user entries provided by a medical provider. The user entries may include, for example, assessments of the member by the medical provider, recommendations by the medical provider to enroll the member for recommended diet and treatment plans, or the like. Other non-limiting examples of the data sources are described with reference to FIG. 5.

In some cases, the server 135 may determine the willingness of a member to be a candidate using AI and/or machine learning techniques described herein. For example, the server 135 may predict the willingness of the member or patient from data records (e.g., stored in provider database 145 and/or patient database 150) that indicate past participation by the member in an assessment and/or personalization program. In some aspects, the data records may include data indicating a prior enrollment of the member in an assessment and/or personalization program.

The server 135 may use one or more contact methods to reach out to members determined as potential candidates for providing a recommended diet and treatment plan. Via the contact methods, the server 135 may recruit the members to download an application (e.g., application 126). In some examples, via the application 126 (and/or browser application 125), the member may enroll to receive an assessment and treatment plan (e.g., diet, lifestyle, titration, etc.).

In some aspects, the server 135 may identify contact methods preferred by the member. For example, the server 135 may identify preferred contact methods from data records (e.g., stored in provider database 145 and/or patient database 150) that indicate contact methods preferred by the member. Additionally, or alternatively, server 135 may identify preferred contact methods based on communication histories between the member and the system 100 (e.g., past communications between the member and a medical provider).

Examples of contact methods supported by the system 100 and identifiable by the system 100 include electronic messages (e.g., email), text messages (e.g., SMS, instant messaging, etc.), voice communications, video and voice communications (e.g., a video call), or the like. The communication devices 105 (e.g., via browser 125 and/or application 126) may support any combination of electronic messages, text messages, voice communications, video and voice communications, or the like.

In an example implementation, the communication device 105 may receive data in an electronic format from the server 135. The data may include an indication that the server 135 has determined (e.g., using the machine learning model(s) 184 and/or targeting logic 191) a member associated with the communication device 105 as a potential candidate for providing a recommended diet and treatment plan. In response to receiving the data, the communication device 105 may display a notification 215 indicating that the member has been identified as a potential candidate. In some examples, the communication device 105 may display one or more buttons 220 (e.g., button 220-a, button 220-b) via which the member may accept or decline to be included in the program.

In some implementations, in response to a user input selecting the button 220-a (‘Accept’), the server 135 may enroll the member in a diet and treatment plan as described herein. In an example, in response to a user input selecting the button 220-a (‘Accept’), the server 135 may request additional information from the member. In an example, the server 135 may request the additional information via a questionnaire 132 described with reference to FIG. 1.

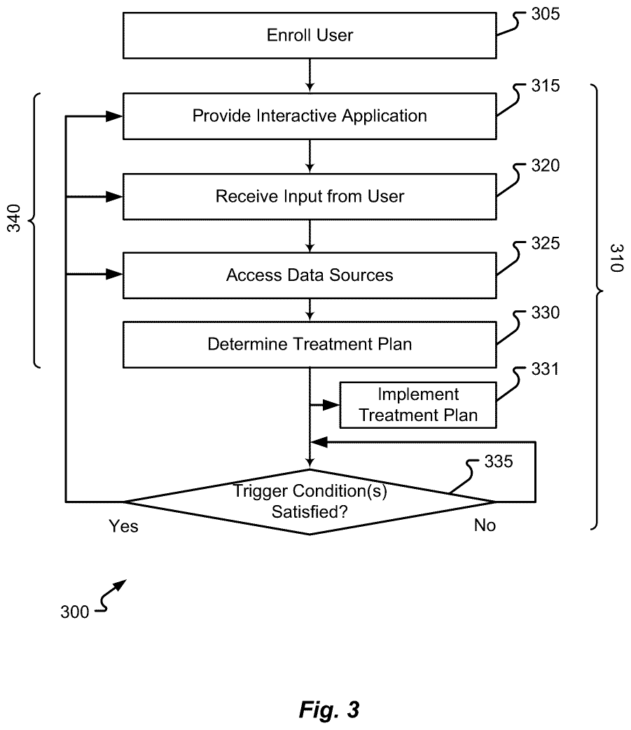

FIG. 3 illustrates an example process flow 300 supportive of diet and deprescription (e.g., a treatment plan) in association with the healthcare management system and digital health platform described herein. Example aspects of process flow 300 may be implemented by a communication device 105 and/or server 135 described with reference to FIG. 1.

At 305, the server 135 may enroll a user in a diet and treatment plan, for example, in response to a user input selecting the button 220-a (‘Accept’) as described with reference to FIG. 2.

At 310, after the user has been enrolled, the server 135 may implement health and nutrition assessment and personalization techniques described herein. For example, at 310, the server 135 may proceed by assessing (e.g., initially assessing) the user's state of health and preferences.

The server 135 may assess the user's state of health and preferences via an interactive application. In an example, the server 135 may provide the interactive application via, for example, a smartphone application (e.g., application 126 of FIG. 1), a website application (e.g., browser application 125 of FIG. 1), and/or some other user interface (e.g., user interface 130 of FIG. 1, a keyboard, a mouse device, etc.). In an example, the interactive application may begin with or include a questionnaire 132 as described with reference to FIG. 1

At 320, the server 135 may receive input from the user in the form of answers to questions (e.g., text inputs, multiple choice selections, etc.) via the interactive application.

At 325, the server 135 may access data sources unique to the system 100. The unique data sources may be, for example, data sources that are associated with or accessible by an entity (e.g., a medical provider, an insurance provider, etc.) but not associated with or accessible by another entity. Non-limiting examples of the data sources are described with reference to FIG. 5.

Based on data accessed by the server 135 from the unique data sources, the server 135 may determine biomarkers for users. In some aspects, based on the data accessed by the server 135 from the unique data sources, the server 135 may determine preferences for users. Example preferences may include preferences with respect to scheduling, coaching, how to be contacted, diet, exercise, lifestyle, or the like, but are not limited thereto.

At 330, based on the user input (at 320) and/or the information accessed (at 325) from the unique data sources, the server 135 may determine an optimal (e.g., best) treatment plan for the individual. In an example, the server 135 may use AI and/or machine learning techniques described herein when determining the optimal treatment plan. For example, using AI and/or machine learning techniques, the server 135 may consider any combination of calories required for the user, nutrients required for the user (e.g., macronutrients, etc.), and state of health of the user in association with determining the optimal treatment plan.

In some aspects, determining the treatment plan at 330 includes determining and balancing an action plan which may enable and support a holistic treatment plan that may include any combination of nutritional treatment, lifestyle management, and medication alteration (e.g., deprescription) described herein, and the like. For example, if the server 135 identifies from the information accessed at 325 that prescribed dosages (e.g., dosage amount, dosage frequency, etc.) for a user have been decreased, the server 135 may determine one or more actions to ensure the user is healthy throughout the deprescription. For example, the server 135 may determine actions (e.g., changes in diet, lifestyle, and/or exercise, etc.) which, if followed by the user, would ensure that the health of the user is maintained or improves throughout the deprescription. In an example, the server 135 may determine changes required to the user's diet and/or lifestyle and may alter suggestions to ensure the user is healthy throughout the deprescription.

At 331, the server 135 may implement the treatment plan as described herein. In some cases, at 331, the server 135 may electronically transmit data indicating the treatment plan to a communication device 105 of medical personnel (e.g., a registered dietician) and/or a communication device 105 of the user.

At 340, the server 135 may redetermine the treatment plan in response to one or more trigger conditions at 335 being satisfied.

For example, the server 135 may determine the treatment plan periodically, such as according to a temporal interval or temporal duration. In another example, the server 135 may determine the treatment plan in response to identifying a prescribed dosage change (e.g., decrease in dosage amount and/or frequency) associated with the user. Accordingly, for example, the server 135 may redetermine the treatment plan in response to one or more of the trigger conditions. The server 135 may repeatedly analyze the current state of health, medical claims/data, and preferences of the user (e.g., via a follow-up questionnaire at 315, receiving additional inputs from the user at 320, additional access of the data sources at 325, etc.), and based on the analysis, the server 135 may maintain or adjust the treatment plan at 330.

In some example implementations, the server 135 may determine the treatment plan using profile matching techniques. For example, the server 135 may analyze a claim history of a member. The server 135 may identify a treatment profile from a set of candidate treatment profiles. The treatment profile may include biometric information (e.g., age, weight, etc.) that matches (e.g., overlaps with) biometric information of the member. In some aspects, the treatment profile may include claim history data (e.g., reason for a medical visit, diagnosis, treatment provided, medication prescribed, etc.) that matches claim history data included in the analyzed claim history of the member. In some cases, the claim history data may include a quantity of visits associated with diagnosing and/or treating a health condition of the member (e.g., quantity of visits associated with diagnosing and/or treating hypoglycemia, hypertension, etc.). In some other aspects, the treatment profile may include lifestyle data (e.g., exercise habits, sleeping habits, smoking habits, etc.) that matches lifestyle data of the member. Accordingly, for example, the server 135 may identify, from the treatment profile, suggested actions (e.g., changes in diet, lifestyle, and/or exercise, etc.) for the member.

As described herein, the server 135 may (1) use the unique data sources in providing personalization for a member, (2) use targeting logic along with algorithms to deprescribe, and (3) develop or alter a treatment plan (e.g., considering macronutrients consumed on a diet in combination with dosage, considering lifestyle alterations in combination with dosage, etc.).

The examples described with reference to FIG. 3 may be applied to the treatment of any medical conditions (e.g., diabetes, hypertension, heart failure, pre-diabetes, weight management, behavioral health, etc.) supported by the healthcare management system described herein.

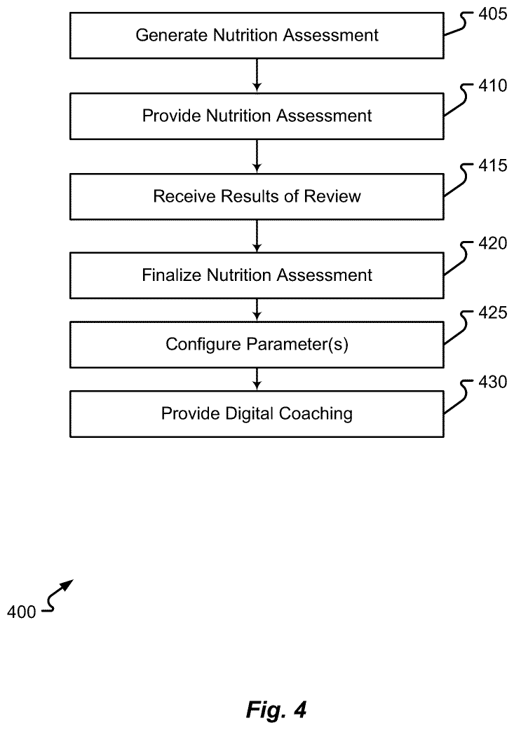

FIG. 4 illustrates an example process flow 400 associated with the healthcare management system and digital health platform described herein. Example aspects of the process flow 400 may be implemented by a communication device 105 and/or a server 135 described with reference to FIG. 1.

Aspects of the process flow 400 support nutrition assessment and personalization and may be implemented by, for example, machine learning model(s) 184 of FIG. 1. In an example, the process flow 400 may couple a nutritional assessment completed by the patient (e.g., questionnaire 132 described with reference to FIG. 1) with a patient's medical history (e.g., based on claims data and prescription data) to recommend personalized diets/nutrition plans to assist and/or guide the patient in managing the medical condition of the patient.

At 405, the server 135 may generate a nutrition assessment personalized for the patient. For example, based on analyzing the answers included in the questionnaire 132, the server 135 may identify: an optimal pattern for managing the medical condition (i.e., a structured diet plan and/or lifestyle plan determined as best for successful deprescription), behavioral barriers associated with the patient which may prevent successful treatment (e.g., successful deprescription), and/or patient preferences indicative of how the patient prefers to be coached. In some aspects, a successful deprescription may include tapering or stopping drugs with respect to a target threshold (e.g., dosage level, dosage frequency, etc.), minimizing polypharmacy (reducing the use of multiple medicines) with respect to a threshold quantity of medication types, and improving a medical condition of a patient with respect to target criteria.

In some aspects, the analysis may include assigning weights to the questions included in the questionnaire 132. For example, aspects of the present disclosure include scoring each question with an integer score (e.g., more or less likely to be successful on a particular nutrition pattern). Accordingly, for example, when analyzing the answers included in the questionnaire 132, the server 135 may assign a weight to each answer in accordance with the weight of the corresponding question.

The questions included in the questionnaire 132 may be customized for different conditions. For example, the questions may include a base question set and a variable question set. The server 135 may select a variable question set for inclusion in the questionnaire 132 based on characteristics of the patient. In an example, for a patient being treated for diabetes using nutritional therapy, lifestyle adjustments, and/or medication alteration, the server 135 may include (as the variable question set) diabetes questions regarding biomarkers and medications.

In some aspects, in identifying behavioral barriers associated with the patient, the behavioral barriers may include a base set of behavioral barriers and a variable set of behavioral barriers. The variable set of behavioral barriers may be based on characteristics of the patient.

At 410, the server 135 may provide the nutrition assessment to a communication device 105 for review by medical personnel (e.g., a registered dietician). For example, the server 135 may electronically transmit the nutrition assessment to the communication device 105. Additionally, or alternatively, the communication device 105 may electronically retrieve the nutrition assessment from the server 135.

At 415, the server 135 may receive results of the review by the medical personnel. The results may include, for example, confirmation of the nutrition assessment (e.g., approval by medical personnel). In some cases, the results may include alterations by the medical personnel to one or more portions of the nutrition assessment provided by the server 135. For example, the results may include alterations (by medical personnel) to any of the pattern for managing the medical condition, behavioral barriers associated with the patient, and patient preferences as provided by the server 135.

At 420, the server 135 may finalize the nutrition assessment generated at 405, based on the results received at 415. For example, the server 135 may maintain all or a portion of the nutrition assessment generated at 405, based on the results received at 415. Additionally, or alternatively, the server 135 may alter all or a portion of the nutrition assessment generated at 405, based on the results received at 415.

At 425, the server 135 may configure one or more parameters for providing digital coaching to the patient, based on the finalized nutrition assessment. For example, the server 135 may configure the application 126 based on the finalized nutrition assessment. In some examples, configuring the application 126 may include configuring communication preferences, alert frequency, encouragement type, message content included in alerts, and the like in association with providing digital coaching to the patient.

At 430, the server 135 may provide digital coaching to the patient (e.g., via a communication device 105, application 126, etc.) based on the finalized nutrition assessment and the configuration of the application 126.

In an example of providing digital coaching, the server 135 may provide the patient with a structured diet plan. The structured diet plan may include a food regimen and an exercise regimen for the patient. In an example, the food regimen may include a diet based on one of low-carbohydrate foods, Mediterranean foods, dietary approaches to stop hypertension (DASH) foods, and plant-based foods that eliminate meat consumption.

The server 135 may communicate the structured diet plan to a communication device 105 of the patient via any combination of notifications (e.g., automated telephone call, email, text message, a notification via the application 126, etc.). In some cases, the server 135 may alert medical personnel to contact (e.g., via a telephone call, email, text message, etc.) the patient in association with encouraging or reminding the patient to adhere to the structured diet plan.

In some example implementations, the server 135 may decrease the frequency in which the server 135 provides a nutrition assessment (as described with reference to 410) for review by medical personnel. For example, the server 135 may decrease the frequency in response to satisfaction of one or more criteria.

In an example, the criteria may include an accuracy percentage of a nutrition assessment generated by the server 135 (at 405) and a nutrition assessment received from medical personnel (at 415) being greater than or equal to a threshold value (e.g., a threshold accuracy). In another example, the criteria may include an overlap amount between a nutrition assessment generated by the server 135 (at 405) and a nutrition assessment received from medical personnel (at 415) being greater than or equal to a threshold value (e.g., an overlap percentage). In some cases, the server 135 may refrain from providing nutrition assessments (as described with reference to 410 through 420) for review by medical personnel in response to satisfaction of the criteria, and the server 135 may proceed without input from medical personnel. Accordingly, for example, the system 100 may support increased automation over time.

FIG. 5 illustrates an example diagram 500 illustrating example data sources 505 processed using AI and machine learning techniques (e.g., using machine learning model(s) 184 of FIG. 1) described herein.

A server 135 may process data accessed from data sources 505 such as an assessment 505-a (e.g., a nutrition assessment, a questionnaire 132 of FIG. 1), medical claims data 505-b, prescription data 505-c, lab data 505-d (e.g., blood pressure, etc.), diagnosis codes 505-c, and peer provided data 505-f. The server 135 may access the data sources 505, for example, from another device (e.g., communication device 105) and/or a database (e.g., a provider database 145 and/or patient database 150 of FIG. 1).

Peer provided data 505-f may include peer-reviewed articles including structured diet plans, provided by medical personnel, in association with treating medical conditions of a patient. For example, peer provided data 505-f may include structured diet plans prescribed by medical personnel for a medical condition (e.g., hemoglobin is elevated, etc.) and results of the structured diet plans with respect to the medical condition.

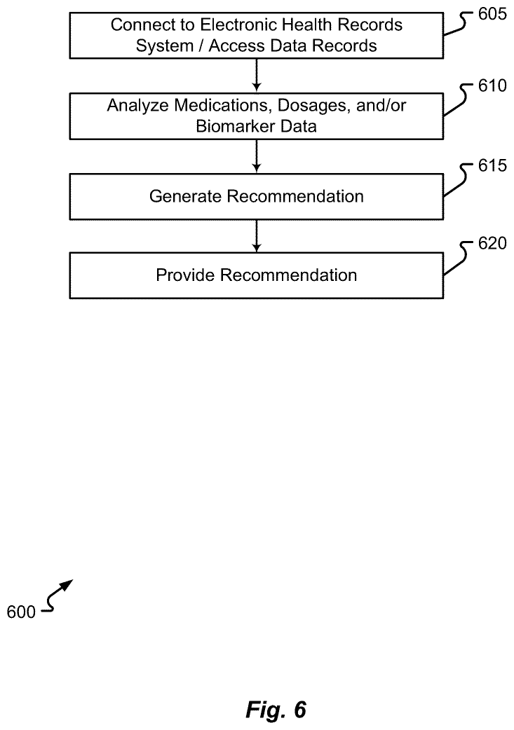

FIG. 6 illustrates an example process flow 600 supportive of deprescription according to aspects of the present disclosure. Example aspects of the process flow 600 may be implemented by a communication device 105 and/or a server 135 described with reference to FIG. 1.

At 605, the server 135 may connect to an electronic health records system (e.g., another server 135, provider database 145, patient database 150, etc.). In an example, at 605, the server 135 may access data records of a patient via the electronic health records system. The server 135 may retrieve, from the data records, current medications of the patient, respective dosages associated with the medications, and historical biomarker data (e.g., blood glucose, blood pressure, heart rate) associated with the patient.

In some aspects, the server 135 may monitor real-time biomarker data (e.g., blood glucose, blood pressure, heart rate, etc.) associated with the patient via one or more sensors. The sensors may be electronically coupled to or integrated with a communication device 105. Additionally, or alternatively, the sensors may be standalone and capable of electronically communicating data with the communication device 105 and the server 135. Examples of the sensors include monitoring devices such as, for example, glucose monitors, blood pressure monitors, heart rate monitors, or the like, and are not limited thereto.

At 610, the server 135 may (e.g., using machine learning model(s) 184) analyze the current medications, respective dosages, and historical biomarker data. In some aspects, the server 135 may determine trends (e.g., patterns, increases, decreases, etc. over time) associated with the medications, respective dosages, and historical biomarker data. Additionally, or alternatively, the server 135 may analyze real-time biomarker data in the analysis and determination of trends.

At 615, the server 135 may generate a recommendation. The recommendation may include nutritional therapy (e.g., a structured diet plan), lifestyle management, and/or medication alteration (e.g., deprescription), based on a result of the analysis.

At 620, the server 135 may provide the recommendation to a medical provider. For example, the server 135 may transmit a message 156 including the recommendation to a communication device 105 associated with the medical provider.

In an example implementation, at 610, the server 135 may analyze current medications, respective dosages, and glucose trends of a patient. For example, the server 135 may determine that, over a 4 day period, glucose levels of the patient moved from 160 mg/dL (on day 1), to 140 mg/dL (on day 2), to 130 mg/dL (on day 3), and then to 110 mg/dL (on day 4). Based on the trend identified by the server 135, the server 135 (at 615) may generate a recommendation including a nutrition alteration, a lifestyle alteration, and/or a medication alteration (e.g., a reduced dosage). At 620, the server 135 may transmit an electronic message (e.g., a treatment update message) to the communication device 105 associated with the medical provider.

In an example, the message may include a recommendation for the medical provider to review and/or decrease the current dosage. In some examples, the message may include the medication alteration (e.g., reduced dosage) generated by the server 135.

In another example, the message may include a recommendation for the medical provider to place the patient on a type of diet (e.g., low-carbohydrate diet, Mediterranean foods, etc.), and the recommendation may include a type of medical titration corresponding to the type of diet. For example, a low-carbohydrate diet may need different insulin levels compared to a Western Pattern Diet (WPD) (also referred to as a Standard American Diet (SAD)).

In another example, the message may include a recommendation for the medical provider to prescribe a lifestyle behavior alteration to the patient. For example, the recommendation may include actions such as increasing the amount of rest (e.g., hours of sleep) per day, increasing the amount of time spent outdoors (e.g., greenspace exposure), refraining from smoking, and the like.

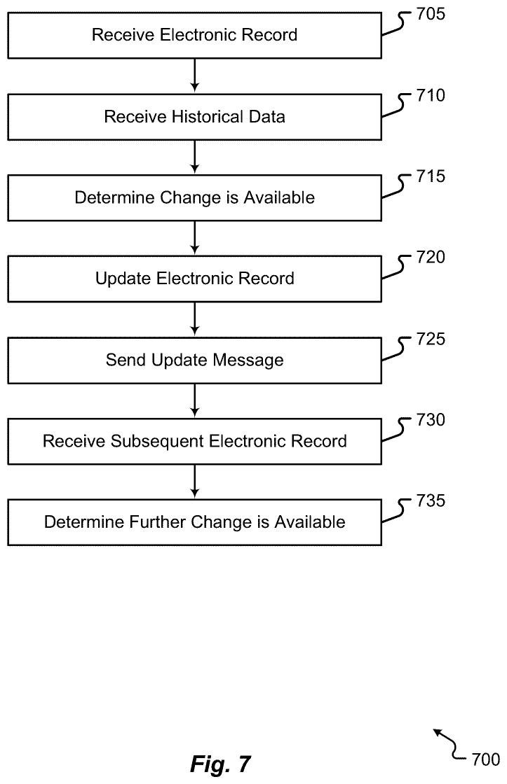

FIG. 7 illustrates an example of a process flow 700 that supports AI based coaching, nutrition therapy, lifestyle management, and medication alteration applicable to treating (e.g., reversing) a medical condition of a member. In some examples, process flow 700 may implement aspects of server 135 or a medical condition management engine 182 described with reference to FIG. 1. In some aspects, elements of the process flow 700 may be implemented based on a user input, autonomously without a user input, and/or semi-autonomously.

In the following description of the process flow 700, the operations may be performed in a different order than the order shown, or the operations may be performed in different orders or at different times. Certain operations may also be left out of the process flow 700, or other operations may be added to the process flow 700.

It is to be understood that while a server 135 and communication devices 105 are described as performing a number of the operations of process flow 700, any device (e.g., a single server 135, another server 135, a combination of servers 135, a single communication device 105, a combination of a server 135 and a communication device 105, etc.) may perform the operations shown.

At 705, the process flow 700 may include receiving an electronic record including health information that is associated with an individual, the health information including fields of the electronic record including prescription-based electronic data, claims-based electronic data, a health condition associated with the individual, a prescribed drug associated with the individual that treats the health condition, a lifestyle behavior associated with the individual, and a structured diet plan associated with the individual.

At 710, the process flow 700 may include receiving historical data about the health information including changes in the health information recorded over time, wherein the changes in the health information include a change in a measured biomarker.

At 715, the process flow 700 may include determining, based on the historical data and the changes in the health information recorded over time received, that a change to at least one of the structured diet plan, the lifestyle behavior, and the prescribed drug is available for the individual.

In some aspects, determining the change to the prescribed drug is available includes determining that a drug titration of the prescribed drug is available for the individual.

In some aspects, determining that the drug titration of the prescribed drug is available for the individual includes: determining, based on a length of time since the individual was initially prescribed the prescribed drug exceeding a predetermined length, that at least one of a drug dependency test and a drug dependency evaluation is recommended for the individual; and at least one of: sending a text message to a communication device of the individual instructing the individual to obtain the drug dependency test; and sending an evaluation message to a communication device of a pharmacy or provider instructing the pharmacy or provider to generate the drug dependency evaluation, wherein the drug dependency evaluation includes an indication of a pattern, a trend, or both associated with the individual and the prescribed drug.

In some aspects, determining that the drug titration of the prescribed drug is available for the individual further includes: receiving results of a drug dependency test associated with the individual; determining, when the results of the drug dependency test indicate the individual is dependent on a minimum dosage of the prescribed drug, that the drug titration will not reach a zero amount of the prescribed drug within a predetermined period of time; and determining, when the results of the drug dependency test indicate the individual is not dependent on the minimum dosage of the prescribed drug, that the drug titration can reach a zero amount of the prescribed drug within the predetermined period of time.

In some aspects, determining that the change to at least one of the structured diet plan, the lifestyle behavior, and the prescribed drug is available for the individual includes: determining, based on the historical data, that a blood measurement of the individual is maintained within predetermined acceptable health levels over a predetermined period of time.

In some aspects, determining that the change to at least one of the structured diet plan, the lifestyle behavior, and the prescribed drug is available for the individual is based on answers provided by the individual in response to score-weighted questions.

At 720, the process flow 700 may include updating the electronic record to store the change to the at least one of the structured diet plan, the lifestyle behavior, and the prescribed drug.

At 725, the process flow 700 may include sending, across a communication network, an update message to a pharmacy or a provider based on determining that the change to at least one of the structured diet plan, the lifestyle behavior, and the prescribed drug is available for the individual.

In some aspects, the update message includes an instruction to alter a parameter associated with at least one of: the structured diet plan, the lifestyle behavior, and the prescribed drug.

In some aspects, the instruction alters at least one of a number of times a prescribed food should be consumed in a given time period, an amount of the prescribed food that should be consumed in the given time period, and a type of the prescribed food that should be consumed in the given time period.

In some aspects, the instruction includes a drug titration to alter the dosage for the prescribed drug associated with the individual, wherein the drug titration decreases at least one of a number of times the prescribed drug should be taken in a given time period and an amount of the prescribed drug that should be taken in the given time period.

In some aspects, the instruction alters at least one of a number of times a prescribed lifestyle action should be performed in a given time period, a duration of the prescribed lifestyle action that should be performed in the given time period, and a type of the prescribed lifestyle action that should be performed in the given time period.

At 730, the process flow 700 may include: receiving, after the individual has adhered to the change to at least one of the structured diet plan, the lifestyle behavior, and the prescribed drug for a predetermined amount of time, a subsequent electronic record including subsequent health information associated with the individual, the subsequent health information including subsequent fields of the electronic record including at least one of blood data and weight information for the individual over the predetermined amount of time.

At 735, the process flow 700 may include determining, based on the subsequent health information, that a further change to at least one of the structured diet plan, the lifestyle behavior, and the prescribed drug is available for the individual.

In some aspects, the further change to the prescribed drug includes a drug titration of the prescribed drug, wherein the drug titration includes reducing an altered dosage amount to zero when the individual is not dependent on a minimum dosage of the prescribed drug.

In some aspects, prior to receiving the electronic record at 705, the process flow 700 includes: determining, based on the health information, the structured diet plan associated with the individual; and sending, across a communication network, the structured diet plan to a communication device of the individual causing the structured diet plan to be rendered by a display of the communication device of the individual.

In some aspects, the structured diet plan is generated by: providing a dataset to a machine learning model, wherein the dataset includes at least one of the prescription-based electronic data and the claims-based electronic data; and receiving an output from the machine learning model in response to the machine learning model processing at least a portion of the dataset, wherein the output includes the structured diet plan.

In some aspects, the dataset further includes at least one of: second prescription-based electronic data associated with the individual; second claims-based electronic data associated with the individual; biomarker data; and answers provided by the individual in response to score-weighted questions.

In some aspects: the structured diet plan is determined based on answers provided by the individual in response to score-weighted questions; the answers are provided in electronic form from a communication device of the individual; and the structured diet plan includes a food regimen, the food regimen including a diet based on one of low-carbohydrate foods, Mediterranean foods, dietary approaches to stop hypertension (DASH) foods, and plant-based foods that eliminate meat consumption.

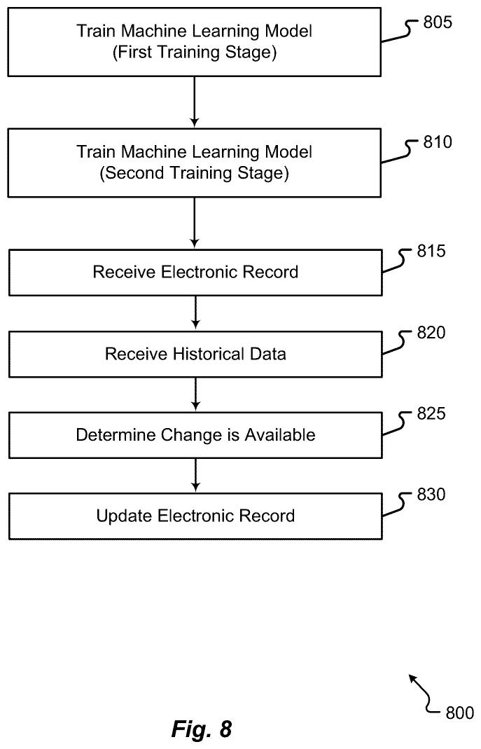

FIG. 8 illustrates an example of a process flow 800 that supports AI based coaching, nutrition therapy, lifestyle management, and medication alteration applicable to treating (e.g., reversing) a medical condition of a member. In some examples, process flow 800 may implement aspects of server 135 or a medical condition management engine 182 described with reference to FIG. 1. In some aspects, elements of the process flow 800 may be implemented based on a user input, autonomously without a user input, and/or semi-autonomously.

In the following description of the process flow 800, the operations may be performed in a different order than the order shown, or the operations may be performed in different orders or at different times. Certain operations may also be left out of the process flow 800, or other operations may be added to the process flow 800.

It is to be understood that while a server 135 and communication devices 105 are described as performing a number of the operations of process flow 800, any device (e.g., a single server 135, another server 135, a combination of servers 135, a single communication device 105, a combination of a server 135 and a communication device 105, etc.) may perform the operations shown.

At 805, the process flow 800 may include training a machine learning model (e.g., machine learning model(s) 184 of FIG. 1) in a first training stage based on a first training set, the first training set including a first set of feature vectors of a first set of individuals for which adherence to one or more prescribed actions achieved one or more relatively positive impacts in association with the health condition.

At 810, the process flow 800 may include training the machine learning model in a second training stage based on a second training set, the second training set including a second set of feature vectors of a second set of individuals for which adherence to the one or more prescribed actions failed to achieve the one or more relatively positive impacts in association with the health condition.

At 815, the process flow 800 may include receiving, at a device, an electronic record including health information that is associated with an individual, the health information including fields of the electronic record including: prescription-based electronic data; claims-based electronic data; a health condition associated with the individual; a prescribed drug associated with the individual that treats the health condition; lifestyle behavior associated with the individual; and a structured diet plan associated with the individual.

At 820, the process flow 800 may include receiving, at the device, historical data about the health information including changes in the health information recorded over time, wherein the changes in the health information include a change in a measured biomarker.

At 825, the process flow 800 may include determining, at the device, based on the historical data and the changes in the health information recorded over time received, that a change to at least one of the structured diet plan, the lifestyle behavior, and the prescribed drug is available for the individual, wherein the change to the prescribed drug includes a drug titration.

In some aspects, determining (at 825) that at least one of the structured diet plan, the lifestyle behavior, and the prescribed drug is available for the individual includes processing at least a portion of the electronic record and at least a portion of the historical data using the machine learning model.

In some aspects, determining (at 825) that at least one of the structured diet plan, the lifestyle behavior, and the prescribed drug is available for the individual includes: providing the electronic record and the historical data to the machine learning model; and receiving an output from the machine learning model in response to the machine learning model processing at least a portion of the electronic record and at least a portion of the historical data, wherein the output includes an indication of the change to at least one of the structured diet plan, the lifestyle behavior, and the prescribed drug.

At 830, the process flow 800 may include updating the electronic record to store the change to the at least one of the structured diet plan, the lifestyle behavior, and the prescribed drug.

A number of implementations have been described. Nevertheless, it will be understood that additional modifications may be made without departing from the scope of the inventive concepts described herein, and, accordingly, other examples are within the scope of the following claims.

The exemplary systems and methods of this disclosure have been described in relation to examples of a communication device 105 and a server 135. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the examples illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed examples, configuration, and aspects.

A number of variations and modifications of the disclosure can be used. It would be possible to provide for some features of the disclosure without providing others.

In yet another example, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another examples, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another example, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the examples with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various examples, configurations, and aspects, includes components, methods, processes, systems and/or apparatus substantially as depicted and described herein, including various examples, subcombinations, and subsets thereof. Those of skill in the art will understand how to make and use the systems and methods disclosed herein after understanding the present disclosure. The present disclosure, in various examples, configurations, and aspects, includes providing devices and processes in the absence of items not depicted and/or described herein or in various examples, configurations, or aspects hereof, including in the absence of such items as may have been used in previous devices or processes, e.g., for improving performance, achieving case, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more examples, configurations, or aspects for the purpose of streamlining the disclosure. The features of the examples, configurations, or aspects of the disclosure may be combined in alternate examples, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed example, configuration, or aspect. Thus, the following claims are hereby incorporated into this Detailed Description, with each claim standing on its own as a separate preferred example of the disclosure.

Moreover, though the description of the disclosure has included description of one or more examples, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative examples, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an example that is entirely hardware, an example that is entirely software (including firmware, resident software, micro-code, etc.) or an example combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A method, comprising:
receiving an electronic record comprising health information that is associated with an individual, the health information comprising fields of the electronic record comprising prescription-based electronic data, claims-based electronic data, a health condition associated with the individual, a prescribed drug associated with the individual that treats the health condition, a lifestyle behavior associated with the individual, and a structured diet plan associated with the individual;
receiving historical data about the health information comprising changes in the health information recorded over time, wherein the changes in the health information comprise a change in a measured biomarker;
determining, based on the historical data and the changes in the health information recorded over time received, that a change to at least one of the structured diet plan, the lifestyle behavior, and the prescribed drug is available for the individual; and
updating the electronic record to store the change to the at least one of the structured diet plan, the lifestyle behavior, and the prescribed drug.

2. The method of claim 1, further comprising:
sending, across a communication network, an update message to a pharmacy or a provider based on determining that the change to at least one of the structured diet plan, the lifestyle behavior, and the prescribed drug is available for the individual.

3. The method of claim 2, wherein the update message comprises an instruction to alter a parameter associated with at least one of: the structured diet plan, the lifestyle behavior, and the prescribed drug.

4. The method of claim 3, wherein the instruction alters at least one of a number of times a prescribed food should be consumed in a given time period, an amount of the prescribed food that should be consumed in the given time period, and a type of the prescribed food that should be consumed in the given time period.

5. The method of claim 3, wherein the instruction comprises a drug titration to alter a dosage for the prescribed drug associated with the individual, wherein the drug titration decreases at least one of a number of times the prescribed drug should be taken in a given time period and an amount of the prescribed drug that should be taken in the given time period.

6. The method of claim 3, wherein the instruction alters at least one of a number of times a prescribed lifestyle action should be performed in a given time period, a duration of the prescribed lifestyle action that should be performed in the given time period, and a type of the prescribed lifestyle action that should be performed in the given time period.

7. The method of claim 1, wherein determining the change to the prescribed drug is available comprises determining that a drug titration of the prescribed drug is available for the individual.

8. The method of claim 7, wherein determining that the drug titration of the prescribed drug is available for the individual comprises:
determining, based on a length of time since the individual was initially prescribed the prescribed drug exceeding a predetermined length, that at least one of a drug dependency test and a drug dependency evaluation is recommended for the individual; and
at least one of:
sending a text message to a communication device of the individual instructing the individual to obtain the drug dependency test; and
sending an evaluation message to a communication device of a pharmacy or provider instructing the pharmacy or provider to generate the drug dependency evaluation, wherein the drug dependency evaluation comprises an indication of a pattern, a trend, or both associated with the individual and the prescribed drug.

9. The method of claim 7, wherein determining that the drug titration of the prescribed drug is available for the individual further comprises:
receiving results of a drug dependency test associated with the individual;
determining, when the results of the drug dependency test indicate the individual is dependent on a minimum dosage of the prescribed drug, that the drug titration will not reach a zero amount of the prescribed drug within a predetermined period of time; and
determining, when the results of the drug dependency test indicate the individual is not dependent on the minimum dosage of the prescribed drug, that the drug titration can reach a zero amount of the prescribed drug within the predetermined period of time.

10. The method of claim 1, wherein determining that the change to at least one of the structured diet plan, the lifestyle behavior, and the prescribed drug is available for the individual comprises:
determining, based on the historical data, that a blood measurement of the individual is maintained within predetermined acceptable health levels over a predetermined period of time.

11. The method of claim 1, further comprising:
receiving, after the individual has adhered to the change to at least one of the structured diet plan, the lifestyle behavior, and the prescribed drug for a predetermined amount of time, a subsequent electronic record comprising subsequent health information associated with the individual, the subsequent health information comprising subsequent fields of the electronic record comprising at least one of blood data and weight information for the individual over the predetermined amount of time; and
determining, based on the subsequent health information, that a further change to at least one of the structured diet plan, the lifestyle behavior, and the prescribed drug is available for the individual.

12. The method of claim 11, wherein the further change to the prescribed drug comprises a drug titration of the prescribed drug, wherein the drug titration includes reducing an altered dosage amount to zero when the individual is not dependent on a minimum dosage of the prescribed drug.

13. The method of claim 1, wherein, prior to receiving the electronic record, the method further comprises:
determining, based on the health information, the structured diet plan associated with the individual; and
sending, across a communication network, the structured diet plan to a communication device of the individual causing the structured diet plan to be rendered by a display of the communication device of the individual.

14. The method of claim 1, wherein the structured diet plan is generated by:
providing a dataset to a machine learning model, wherein the dataset comprises at least one of the prescription-based electronic data and the claims-based electronic data; and
receiving an output from the machine learning model in response to the machine learning model processing at least a portion of the dataset, wherein the output comprises the structured diet plan.

15. The method of claim 14, wherein the dataset further comprises at least one of:
second prescription-based electronic data associated with the individual;
second claims-based electronic data associated with the individual;
biomarker data; and
answers provided by the individual in response to score-weighted questions.

16. The method of claim 1, wherein:
the structured diet plan is determined based on answers provided by the individual in response to score-weighted questions;
the answers are provided in electronic form from a communication device of the individual; and
the structured diet plan comprises a food regimen, the food regimen comprising a diet based on one of low-carbohydrate foods, Mediterranean foods, dietary approaches to stop hypertension (DASH) foods, and plant-based foods that eliminate meat consumption.

17. The method of claim 1, wherein determining that the change to at least one of the structured diet plan, the lifestyle behavior, and the prescribed drug is available for the individual is based on answers provided by the individual in response to score-weighted questions.

18. A system for reducing medication dependency, comprising:
a communications interface;
a processor coupled with the communications interface; and
a memory coupled with the processor, wherein the memory stores data that, when executed by the processor, enables the processor to:
receive, via the communications interface, an electronic record comprising health information that is associated with an individual, the health information comprising fields of the electronic record comprising prescription-based electronic data, claims-based electronic data, a health condition associated with the individual, a prescribed drug associated with the individual that treats the health condition, a lifestyle behavior associated with the individual, and a structured diet plan associated with the individual;
receive, via the communications interface, historical data about the health information comprising changes in the health information recorded over time, wherein the changes in the health information comprise a change in a measured biomarker;
determine, based on the historical data and the changes in the health information recorded over time received, that a change to at least one of the structured diet plan, the lifestyle behavior, and the prescribed drug is available for the individual; and
update the electronic record to store the change to the at least one of the structured diet plan, the lifestyle behavior, and the prescribed drug.

19. The system of claim 18, wherein the data, when executed by the processor, further enables the processor to:
send, via the communications interface, an update message to a pharmacy or a provider based on determining that the change to at least one of the structured diet plan, the lifestyle behavior, and the prescribed drug is available for the individual.

20. The system of claim 19, wherein the update message comprises an instruction to alter a parameter associated with at least one of: the structured diet plan, the lifestyle behavior, and the prescribed drug.

21. The system of claim 19, wherein determining the change to the prescribed drug is available comprises determining that a drug titration of the prescribed drug is available for the individual,
wherein in determining that the drug titration of the prescribed drug is available for the individual, the data, when executed by the processor, further enables the processor to:
determine, based on a length of time since the individual was initially prescribed the prescribed drug exceeding a predetermined length, that at least one of a drug dependency test and a drug dependency evaluation is recommended for the individual; and
at least one of:
send, via the communication interface, a text message to a communication device of the individual instructing the individual to obtain the drug dependency test; and
send, via the communication interface, an evaluation message to a communication device of a pharmacy or provider instructing the pharmacy or provider to generate the drug dependency evaluation, wherein the drug dependency evaluation comprises an indication of a pattern, a trend, or both associated with the individual and the prescribed drug.

22. A computer-implemented method, comprising:
receiving, at a device, an electronic record comprising health information that is associated with an individual, the health information comprising fields of the electronic record comprising:
prescription-based electronic data;
claims-based electronic data;
a health condition associated with the individual;
a prescribed drug associated with the individual that treats the health condition;
lifestyle behavior associated with the individual; and
a structured diet plan associated with the individual;
receiving, at the device, historical data about the health information comprising changes in the health information recorded over time, wherein the changes in the health information comprise a change in a measured biomarker;
determining, at the device, based on the historical data and the changes in the health information recorded over time received, that a change to at least one of the structured diet plan, the lifestyle behavior, and the prescribed drug is available for the individual, wherein the change to the prescribed drug comprises a drug titration; and
updating the electronic record to store the change to the at least one of the structured diet plan, the lifestyle behavior, and the prescribed drug.

23. The computer-implemented method of claim 22, further comprising:
training a machine learning model in a first training stage based at least in part on a first training set, the first training set comprising a first set of feature vectors of a first set of individuals for which adherence to one or more prescribed actions achieved one or more relatively positive impacts in association with the health condition; and
training the machine learning model in a second training stage based at least in part on a second training set, the second training set comprising a second set of feature vectors of a second set of individuals for which adherence to the one or more prescribed actions failed to achieve the one or more relatively positive impacts in association with the health condition,
wherein determining that at least one of the structured diet plan, the lifestyle behavior, and the prescribed drug is available for the individual comprises processing at least a portion of the electronic record and at least a portion of the historical data using the machine learning model.

24. The computer-implemented method of claim 22, wherein determining that at least one of the structured diet plan, the lifestyle behavior, and the prescribed drug is available for the individual comprises:
providing the electronic record and the historical data to a machine learning model; and
receiving an output from the machine learning model in response to the machine learning model processing at least a portion of the electronic record and at least a portion of the historical data, wherein the output comprises an indication of the change to at least one of the structured diet plan, the lifestyle behavior, and the prescribed drug.
